(19) United States
(12) Patent Application Publication
(10) Pub. No.: US 2010/0167317 A1
(43) Pub. Date: Jul. 1, 2010

(54) PROCESS FOR ENRICHING BASOPHILS IN A BLOOD SAMPLE

(75) Inventors: Marc DAERON, Chevreuse (FR); Nihad MEKNACHE, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: INSTITUT PASTEUR, Paris (FR)

(21) Appl. No.: 12/534,272

(22) Filed: Aug. 3, 2009

Related U.S. Application Data
(60) Provisional application No. 61/086,228, filed on Aug. 5, 2008.

(51) Int. Cl. G01N 33/53; C12N 5/078

(52) U.S. Cl. 435/7.24; 435/372

(57) ABSTRACT

The present invention is based on the discovery that contrary to prior studies CD16 is expressed by basophils and as such provide methods and kits for enriching basophils in a blood sample.

### CROSS REFERENCE TO RELATED APPLICATIONS

This application claims the benefit of U.S. provisional application 61/086,228 filed Aug. 5, 2008.

### BACKGROUND OF THE INVENTION

1. Field of the Invention

The present invention is based on the discovery that contrary to prior studies CD16 is expressed by basophils and as such provides methods and kits for enriching basophils in a blood sample.

2. Description of the Related Art

Basophils are increasingly recognized as major players in allergy. Like mast cells, they contain and, upon stimulation, they release preformed granular vasoactive amines, they synthesize lipid-derived mediators and they secrete high amounts of Th2 cytokines (Gibbs, B. F. 2005. Clinical and experimental medicine 5:43-49). As a consequence, basophils are thought to play critical roles not only as the effectors of acute reactions such as anaphylaxis, but also, by promoting Th2 polarization, as the inducers of an atopic phenotype (Min, B., and W. E. Paul. 2008. Current opinion in hematology 15:59-63). Besides, basophils were recently shown to promote IgE-dependent chronic allergic inflammation in mice (Mukai, K et al 2005. Immunity 23:191-202).

Basophils can be activated by numerous extracellular stimuli which engage cell-activating receptors. Among these are receptors for the Fc portion of antibodies (FcRs). Activating FcRs include IgE and IgG receptors. Basophils express high-affinity IgE receptors (FcεRI), which are well known as the triggers of IgE-dependent allergic reactions and anaphylaxis (Turner, H., and J. P. Kinet. 1999. Nature 402:B24-30). The activating properties of FcεRI lie on the presence of Immunoreceptor Tyrosine-based Activation Motifs (ITAMs) in the intracytoplasmic domains of FcRγ and FcRβ, the two FcR common subunits with which they are constitutively associated (Daëron, M. 1997. Annu. Rev. Immunol. 15:203-234). Mast cells and basophils also express low-affinity IgG receptors. Mouse, but not human mast cells express FcγRIIIA which also associate with FcRγ and FcRβ in these cells. FcγRIIIA activate mouse mast cells, both in vitro (Daëron, 1992. J. Immunol. 149:1365-1373) and in vivo (Hazenbos, et al 1996. Immunity 5:181-188). Whether mouse basophils express FcγRIIIA is not known. Basophils were however recently reported to account for IgG-induced, but not IgE-induced passive systemic anaphylaxis, whereas mast cells account for IgE-induced, but not IgG-induced anaphylaxis in mice (Tsujimura, et al 2008. Immunity 28:581-589). Mouse basophils therefore express functional activating IgG receptors. Whether a similar situation applies to humans is not known. Human, but not mouse, mast cells and basophils express FcγRIIA/C (Daëron, et al 1995. Immunity 3:635-646), which do not associate with FcRγ or FcRβ, but which contain an ITAM in their own intracytoplamsic domain. FcγRIIA were shown to activate mast cells in RBL-2H3 transfectants (Daëron, et al 1995. Immunity 3:635-646) and in skin-derived human mast cells (Zhao et al 2006. J Immunol 177:694-701). Whether they can activate human basophils is not known. Finally, although not formally demonstrated, human basophils probably also express inhibitory IgG receptors (FcγRIIB). The co-engagement of IgG receptors with IgE receptors on basophils by allergen-IgG antibody complexes was indeed found to inhibit IgE-induced histamine release (Daëron, et al 1995. Immunity 3:635-646, Tam, et al 2004. Allergy 59:772-780), as it had been previously shown in mouse mast cells (Daëron et al 1995. J. Clin. Invest. 95:577-585). FcγRs are therefore potential positive and/or negative regulators of basophil activation. Basophils FcγRs are however poorly characterized. We therefore undertook an investigation of FcγRs expressed by human basophils in normal and allergic individuals.

Studies on basophils have been hampered by the low number of these cells in peripheral blood and, due to the unavailability of a basophil-specific growth factor, by the lack of reliable in vitro model. Various methods of basophil enrichment have therefore been developed, using centrifugation (e.g. elutriation) and/or sorting procedures based on the differential expression of membrane molecules by basophils. These enrichment techniques are not devoid of inconvenience. Basophils may be altered (e.g. activated) by positive selection procedures while some may be discarded by negative selection procedures. In order to avoid any such bias, we used unfractionated white blood cells, among which basophils were identified by flow cytometry using CD203c. Differing from other membrane molecules, CD203c is indeed a specific marker of basophils (Reimer et al 2006. Allergy 61:1063-1070). It is constitutively expressed and upregulated upon basophil activation (Buhring et al 2004. Int Arch Allergy Immunol 133:317-329). CD203c can therefore be used as a positive criterion for identifying basophils, and its increased expression as an indicator of basophil activation.

Classically, basophils also express CD32, but not CD16 (Reimer et al 2006. Allergy 61:1063-1070, Takahashi et al 1993. J Immunol Methods 162:17-21, Han et al 1999 Cytometry 37:178-183). The CD nomenclature is indeed often used to designate human FcRs. CD32 refers to FcγRII whereas CD16 refers to FcγRIII. CD32 therefore includes FcγRIIA, FcγRIIB and FcγRIIC, i.e. one inhibitory and two activating receptors. One mAb specifically recognizes FcγRIIA, but there is no commercially available FcγRIIB- or FcγRIIC-specific antibody. Likewise, CD16 includes two FcγRIII isoforms encoded by two distinct genes. FcγRIIIA (CD16A) is a FcRγ-associated (Ra, C et al 1989. Nature 341:752-754) transmembrane receptor (Ravetch, J. V., and B. Perussia. 1989. J. Exp. Med. 170:481-497) expressed by monocytes (Klaassen et al 1990 J Immunol 144:599-606), NK cells (Anderson, et al 1990 Proc. Natl. Acad. Sci. USA 87:2274-2278) and NKT cells (Kim, et al 2006. J Clin Invest 116:2484-2492). FcγRIIIB (CD16B) is a glycosylphosphatidylinositol (GPI)-anchored receptor which does not associate with FcRγ and has no intracytoplasmic domain (Lanier, et al 1989 Science 246:1611-1613). CD16B is thought to be exclusively expressed by human polymorphonuclear neutrophils (Li et al 1996 J. Exp. Med. 183:1259-1263). No FcγRIIIA- or FcγRIIIB-specific antibody is available.

### SUMMARY OF THE INVENTION

We found that blood basophils express FcγRII/CD32 as expected, but also FcγRIII/CD16. We demonstrate that basophils express CD16B, but not CD16A, that CD16B is detectably expressed by at least one fourth of basophils in all normal donors.

On the basis of this discovery, one embodiment of the invention is a method of enriching basophils in a blood sample by contacting the sample with antibodies that bind to non-basophil cells, excluding any anti-CD16 antibody and separating or removing those non-basophil cells.

Another embodiment of the invention is a kit that contains one or more antibody cocktails that bind to the non-basophil cells, excluding any anti-CD16 antibody.

### BRIEF DESCRIPTION OF THE DRAWINGS

FIG. 1. Identification of basophils by flow cytometry. Left panel: Representative FSC/SSC dot plot of RBC-depleted blood cells from a normal donor. Ovals show regions containing neutrohils, lymphocytes and monocytes, respectively. The rectangle shows the gate used for basophils. Middle panel: The same cells were incubated with normal Goat IgG-FITC and IgG1 679.1Mc7-PE. FL1/FL2 dot plot. Right panel: The same cells were incubated with Goat IgG anti-human IgE-FITC and CD203-PE. FL1/FL2 dot plot.

FIG. 2. Basophils express FcγRII/CD32 and FcγRIII/CD16. (A) Cells were incubated with CD14-PE, CD24-PE, CD56-PE, CD19-PE, CD3-PE, CD203-PE or PE-conjugated isotype controls, and with CD32-FITC, CD16-FITC or FITC-conjugated isotype controls. (B) Cells were incubated with CD203-PE, Goat anti-human IgE-FITC and CD16-APC. FL1/FL2 dot plot. CD16⁺ cells are shown in red.

FIG. 3. Basophils express GPI-anchored FcγRIIIB/CD16B. (A) Cells were treated or not with PI-PLC, and labeled with CD16-FITC and CD203-PE, CD24-PE, CD14-PE or CD56-PE. Figures are FL1-MFIs in gates shown in bold. (B) Cells were treated or not with PI-PLC, and labeled with CD32-FITC and CD203-PE, CD24-PE, or CD14-PE. Figures are FL1-MFIs in gates shown in bold.

FIG. 4. CD16 expression by basophils is decreased upon anti-IgE-induced activation. (A) Cells were stimulated with anti-IgE or with CD16 F(ab′)₂+RAM F(ab′)₂, and labeled with CD203-PE. Figures are FL2-MFI values measured in gates shown in bold. (B) Cells from 8 normal donors were stimulated with anti-IgE antibodies or without, and labeled with CD203-PE (left) or CD16-FITC (right).

FIG. 5. All donors have CD16⁺ basophils and the percentage of CD16⁺ basophils is reduced in atopic dermatitis patients. Cells from 23 normal donors and 25 patients were labeled with CD16-FITC and CD203-PE. Symbols represent the percentage of basophils (CD203⁺ cells) stained by CD16 over background in individual subjects.

FIG. 6. The percentage of CD16⁺ basophils is not correlated with gender, age, or CD203 expression. Percentages of CD16⁺ basophils in patients (A) as a function of gender and, (B) as a function of age. (C) CD203 expression in the same normal donors and patients as in FIG. 5.

FIG. 7. The percentage of CD16⁺ basophils is not correlated with serum IgE. Serum IgE levels in patients (A) as a function of gender and, (B) as a function of age. (C) Percentages of CD16⁺ basophils in the same patients as in FIG. 5, as a function of serum IgE concentration.

### DETAILED DESCRIPTION OF THE PREFERRED EMBODIMENTS

Although a minor population in blood, basophils play major roles in allergy. Basophils are well known to express high-affinity IgE receptors (FcεRI). They also express low-affinity IgG receptors which are ill-defined. Low-affinity IgG receptors comprise CD32 (FcγRIIA, FcγRIIB and FcγRIIC), and CD16 (FcγRIIIA and FcγRIIIB). FcγRIIA, FcγRIIC and FcγRIIIA are activating receptors, FcγRIIB is an inhibitory receptor, FcγRIIIB is a glycosylphosphatidylinositol-anchored receptor whose function is poorly understood. Basophils were reported to express CD32, but not CD16.

This work aimed at identifying IgG receptors on blood basophils. Basophils from normal donors and from patients suffering from an allergic skin disease (atopic dermatitis) allergic respiratory diseases (rhinitis and allergic asthma) or a non-allergic skin disease (chronic urticaria) were examined. We found that 24-81% basophils from normal donors express CD16, that basophils express FcγRIIIB/CD16B, but not FcγRIIIA/CD16A, that CD16 expression is downregulated upon anti-IgE-induced basophil activation, and that the proportion of CD16⁺ basophils is significantly decreased in atopic dermatitis patients. Our results challenge the two dogmas 1) that basophils do not express CD16 and 2) that CD16B is exclusively expressed by neutrophils. They suggest that a proportion of basophils may be lost by enrichment procedures in which CD16⁺ cells are discarded. They unravel an unexpected complexity of IgG receptors susceptible to modulate basophil activation. They identify a novel systemic alteration in atopic dermatitis.

A high degree of basophil purity is often required for functional studies with basophils, especially when mediator secretion from contaminating cells masks what is released by basophils. Pure basophil populations are also mandatory when investigating either intracellular signaling events or to exclude the possibility of priming cytokines (eg, IL-3, GM-CSF), derived from cellular contaminants, to influence basophil function. Therefore, several protocols for basophil purification have been published over the last decade (see references in Haisch et al J Immunol Methods. 1999; 226:129-137). When purifying basophils, one is faced with several challenges. First, the average percentage of basophils in the peripheral blood of healthy individuals is low (<1%), which restricts the possible yield. Furthermore, a large donor variation in the proportion of basophils makes the final recovery of basophils unpredictable, especially when (dealing with blood from unknown donors (ie, from buffy coats) (Gibbs. Inflamm Res. 1997; 46:137-142).

Most of the currently published purification techniques rely on initial physical separation methods (density gradients or elutriation) that, by themselves, do not reliably result in very high purity but are successful in enriching basophils sufficiently enough for further purification by immunoselection. Some protocols have relied on the expression of the high-affinity receptor FcRI on the surface of human basophils (Schroeder J T, Hanrahan L R J. Purification of human basophils using mouse monoclonal IgE. J Immunol Methods. 1990; 133:269-277 Weil G J, Leiserson W M, Chused T M. Isolation of human basophils by flow microfluorometry. J Immunol Methods. 1983; 58:359-363). This positive selection technique, although resulting in very high basophil purity, is not desirable for many purposes because of the likelihood of activation during purification. The use of negative selection with magnetic beads (Kepley C L, Pfeiffer J R, Schwartz L B, Wilson B S, Oliver J M. The identification and characterization of umbilical cord blood-derived human basophils. J Leukoc Biol. 1998; 64:474-483; Mul F P, Knol E F, Roos D. An improved method for the purification of basophilic granulocytes from human blood. J Immunol Methods. 1992; 149:207-214; Bjerke T, Nielsen S, Helgestad J, Nielsen B W, Schiotz P O. Purification of human blood basophils by negative selection using immunomagnetic beads. J Immunol Methods. 1993; 157:49-56) consistently resulted in viable, functional (nonpreactivated) basophils but, in our experience, high yields were usually obtained only with hyperbasophilic donors (Gibbs. Inflamm Res. 1997; 46:137-142). A recently published protocol, however, has overcome this limitation and uses a commercially available kit, (Haisch K, Gibbs B F, Korber H, et al. Purification of morphologically and functionally intact human basophils to near homogeneity. J Immunol Methods. 1999; 226:129-137) therefore making the purification protocol widely accessible to any laboratory with an interest in granulocyte function. A similar protocol combines density gradient centrifugation with negative selection with magnetic beads (Tsang S, Hayashi M, Zheng X, Campbell A, Schellenberg R R. Simplified purification of human basophils. J Immunol Methods. 2000; 233:13-20).

The discovery of the inventors that basophils express one of the isoform of IgG receptor CD16, CD16B, have led them to provide a new basophil enriching method by negative immunoselection.

The method for enriching basophils according to the invention in a blood sample consists in depleting the blood sample of non basophils. This is accomplished by contacting the sample with an antibody cocktail that comprises antibodies that specifically bind to non-basophils, and separating the non-basophils from the sample to obtain a sample enriched with basophils, wherein antibodies specific for CD16 are excluded of used antibodies. Preferably, antibodies specific for CD16B are excluded, i.e., not used.

Antibodies that specifically bind to non-basophils comprise one or more antibodies which specifically bind to neutrophils, one or more antibodies which specifically bind to natural killer cells, one or more antibodies which specifically bind to monocytes, one or more antibodies which specifically bind to dendritic cells, one or more antibodies which specifically bind to T lymphocytes, one or more antibodies which specifically bind to B lymphocytes, and one or more antibodies which specifically bind to eosinophils.

Optionally antibodies that specifically bind to non-basophils can further comprise one or more antibodies which specifically bind to erythrocytes and one or more antibodies which specifically bind to platelets.

In a preferred embodiment of the invention, the antibody cocktail comprises one or more antibodies specific for CD14, one or more antibodies specific for CD24, and one or more antibodies specific for CD56.

In a more preferred embodiment of the invention, the antibody cocktail comprises:

at least one antibody specific for CD3,

at least one antibody specific for CD4,

at least one antibody specific for CD7,

at least one antibody specific for CD14,

at least one antibody specific for CD15,

at least one antibody specific for CD24,

at least one antibody specific for CD36,

at least one antibody specific for CD45RA,

at least one antibody specific for HLA-DR,

at least one antibody specific for CD56, and

at least one antibody specific for CD235a.

In another more preferred embodiment of the invention, the antibody cocktail comprises:

at least one antibody specific for CD2,

at least one antibody specific for CD3,

at least one antibody specific for CD14,

at least one antibody specific for CD15,

at least one antibody specific for CD19,

at least one antibody specific for CD24,

at least one antibody specific for CD34,

at least one antibody specific for CD36,

at least one antibody specific for CD45RA,

at least one antibody specific for CD56, and

at least one antibody specific for CD235a.

Antibodies used in the method for enriching basophils, can be monoclonal or polyclonal and can be obtained according to standard antibody technology known in the art. Indeed, the antigens listed herein are known from a variety of mammalian organisms, including humans and antibodies that specifically bind to these antigens are either available commercially or readily obtained by known methods.

Preferably monoclonal antibodies are used for carrying out the method according to the invention. More preferably, antibodies used in the present invention are monoclonal antibodies of IgG type.

As used herein, the terms “immunoglobulin” and “antibody” refer to a protein consisting of one or more polypeptides substantially encoded by immunoglobulin genes. Immunoglobulins may exist in a variety of forms besides antibodies, including for example, Fv, Fab, and F(ab)₂, as well as in single chains.

Antibodies may be produced in a variety of ways. The production of non-human monoclonal antibodies, e.g., murine, lagomorpha, equine, etc., is well known and may be accomplished by, for example, immunizing the animal with a preparation containing the polypeptide. Antibody-producing cells obtained from the immunized animals are immortalized and screened. Methods of producing polyclonal and monoclonal antibodies are known to those of skill in the art. See, e.g., Coligan (1991) Current Protocols in Immunology Wiley/Greene, N.Y.; and Harlow and Lane (1989) Antibodies: A Laboratory Manual Cold Spring Harbor Press, N.Y. Specific monoclonal and polyclonal antibodies will usually bind with a Kd of at least about 0.1 mM, more usually at least about 1 μM, and most preferably at least about 0.1 μM or better.

Generally, diluting antibodies in a buffer and storing antibodies is known however, those dilutions typically were performed in phosphate buffered saline or other isotonic solution and may also have small quantities of bovine serum albumin and in some instances relatively small amounts of detergents such as Triton X-100 or NP-40 (Antibodies: A Laboratory Manual, Harlow and Lane, Cold Spring Harbor Laboratory Press, Cold Spring Harbor, N.Y., 1988; and Current Protocols in Molecular Biology, Ausebel et al (eds.), John Wiley and Sons, Inc. N.Y., 2001).

Antibodies used in the present invention can be used singly or in combination with two or more antibodies for the same or different epitopes on the protein being targeted. The antibodies can be different in that the antibodies react with different antigens and/or different isoforms or epitopes of a particular protein/antigen in a single sample.

Conjugating detectable moieties or enzyme systems to antibodies is known and may be used in the present invention. Anti-non basophils antibodies can also be conjugated (or coupled) to a solid support. Preferably, antibodies are conjugated to magnetic polymer as Dynal® beads or Microbeads®, the obtained beads are commonly named immunomagnetic beads. After incubation with the blood sample, the immunomagnetic beads are retained by applying a magnetic field, several magnetic sorters are commercialized: magnetic particle concentrator MPC-1 by Dynal®, MACS® column and a MACS® Separator by Miltenyi Biotec.

In another preferred embodiment, anti-non basophils antibodies are conjugated with biotin, in this case anti-biotin monoclonal antibodies conjugated to magnetic beads are used as secondary labeling reagent.

In another embodiment, the anti-non basophils antibodies (for example IgG) can also be unlabeled, in this case magnetically labeled antibodies directed against these antibodies (anti-IgG antibodies) are used as secondary labeling reagent.

An “individual” or “patient” which may be subjected to the methodology described herein may be any of mammalian animals including human, dog, cat, cattle, goat, pig, swine, sheep and monkey. Humans are preferable.

A “sample” is a biological sample such as blood, serum, lymph, and tissue. The “sample” may also be pretreated, for example, by homogenization, extraction, enzymatic and/or chemical treatments as commonly used in the field. As used herein, blood sample refers to peripheral blood, buffy coat, umbilical cord blood or peripheral blood mononuclear cells (PBMC). Buffy coat is a fraction of an anticoagulated blood sample depleted of erythrocytes and plasma by density gradient centrifugation. Peripheral blood mononuclear cells (PBMC) can be obtained by density gradient centrifugation of blood, for example by Ficoll density gradient centrifugation.

Fresh blood sample is usually treated with an anti-coagulant (e.g. heparin, EDTA, citrate, ACD-A, or CPD).

The blood sample can be pretreated before applying the basophils enriching method. Several pre-treatments are routinely used to enrich sample in mononuclear cells, for example Ficoll density centrifugation (Gibbs et al., Inflamm. Research 1997; 46:137-142; Ficoll Hypaque® separation commercialized by Amersham Pharmacia Biotech), counter current centrifugal elutriation (Gibbs et al., Inflamm. Research 1997; 46:137-142), Percoll density gradient centrifugation (Tanimoto et al. Clin. Exp. Allergy 1992; 22: 1015-1019; Bjerke et al. J. Immunol. Meth 1993; 157:49-56). To remove contaminating erythrocytes from sample, the sample can be incubated with lysis buffer (155 mM NH₄Cl, 10 mM KHCO₃ and 0.1 mM EDTA).

Non-basophilic cells are known in the art. Non-basophils or non-basophilic cells are T-cells, Natural Killer (NK) cells, B cells, monocytes, dendritic cells, erythroid cells, platelets, neutrophils, and eosinophils.

Separating the non-basophils from the sample to obtain a sample enriched with basophils can be accomplished by different ways well known to those skill in the art. As preferred embodiments, several separating steps are described below.

When the anti-non basophils antibodies are fixed on a solid carrier, the blood sample, after incubating with antibodies, is simply recovered.

When the anti-non basophils antibodies are conjugated to a magnetic support as magnetic beads, applying a magnetic field permits to deplete the magnetically labeled non-basophils by retaining them on the magnetic sorter, on a column for example, while the unlabeled basophils pass through.

When the anti-non basophils antibodies are conjugated to biotin, after contacting the sample with anti-non basophils antibodies, separating consists in contacting the sample with anti-biotin antibodies conjugated to a magnetic support as magnetic beads and applying a magnetic field to deplete the magnetically labeled non-basophils.

When the anti-non basophils antibodies are unconjugated, separating can consist in contacting the sample with secondary antibodies (anti-anti-non basophils antibodies) conjugated to a magnetic support, and applying a magnetic field to deplete the magnetically labeled non-basophils.

In an embodiment of the invention, the separating step may be repeated one or more times.

Optionally, the method according to the invention further comprises evaluating the purity of the enriched basophils. The purity of the enriched basophils can be evaluated by flow cytometry or fluorescence microscopy. The sample can be stained with the basophil marker CD203c. Alternatively, basophils can be visualized as CD123^{hi} CD303 (BDCA2)⁻ cells by staining with CD303 and CD123 antibodies. Basophils analyze can be performed by flow cytometry or fluorescence microscopy. Labeling of non-basophils with the biotin-antibody cocktail can be visualized by counterstaining with a fluorochrome-conjugated anti-biotin antibody (e.g. anti-biotin-PE or anti-biotin-APC).

In certain aspects of the invention, utilizing the method for enriching basophils according to the invention permits to achieve a purity of blood basophils consistently greater than 95% with yields 24 to 81% superior to yields obtained with the classical negative selection immunomagnetic methods.

The invention also provides a kit or kits that can be used to facilitate the methods described herein above. Such a kit can include antibodies or functional antibody fragments, e.g., Fab or Fc etc. that specifically bind to non-basophils and, preferably are conjugated to magnetic solid supports such as magnetic beads. The antibodies or functional antibody fragments can be provided separately or cocktailed in one or more combinations of antibodies. For example, all antibodies or antibody fragments can be mixed in a single antibody cocktail.

The antibodies or functional antibody fragments can be provided, individually or cocktailed can be provided in concentrated form, which can then be diluted by the user or pre-diluted. The antibodies or functional antibody fragments can be provided diluted, for example, at least 1:50 and preferably from about 1:50 to 1:6000 and all ranges and values therebetween.

According to the invention, the antibodies or functional antibody fragments can be provided in a composition comprising a buffer or buffer system that stabilizes the antibodies and/or enables their use for enriching basophils in/from a sample. The kit can also include separate buffers that the user can employ to dilute the antibodies or functional antibody fragments.

The antibodies or functional antibody fragments can be provided already conjugated to a solid support or can be provided not conjugated. Combinations of these can also be employed, e.g., one or more antibodies or functional antibody fragments can be provided pre-conjugated and other antibodies or functional antibody fragments can be provided unconjugated. In some embodiments, if the antibodies or functional antibody fragments are not preconjugated, then reagents and materials (e.g., solid supports) can be included in the kit for the user to conjugate the antibodies or functional antibody fragments prior to their use.

In some embodiments, the kit can be packaged with reagents and/or devices that enable the enrichment of basophils, i.e., depleting non-basophilic cells from the sample or isolating basophils. For example, for many solid supports employed in the field, columns can be used to accomplish this. Thus, the kit can include such reagents and/or devices to facilitate the recovery and/or depletion of cells as needed.

Instructions for use of the components of the kit can also be provided.

According to the invention, the antibodies or functional antibody fragments, provided individually or cocktailed into one or more antibody cocktails includes antibodies or functional antibody fragments which specifically bind to non basophils surface markers, except CD16 and preferably except CD16B.

In a preferred embodiment, the antibodies or functional antibody fragments includes antibodies or functional antibody fragments which specifically bind CD14, CD24, and CD56, and in a more preferred embodiment includes antibodies or functional antibody fragments which specifically bind to CD3, CD4, CD7, CD14, CD15, CD24, CD36, CD45RA, HLA-DR, CD56, and CD235a; or in another more preferred embodiment includes antibodies or functional antibody fragments which specifically bind to CD2, CD3, CD14, CD15, CD19, CD24, CD34, CD36, CD45RA, CD56, and CD235a.

### EXAMPLES

### Materials and Methods

Subjects. A) Healthy controls: Blood from normal donors was obtained from the Centre Necker-Cabanel of the Etablissement Français du Sang, (Paris, France). B) Patients: Peripheral blood was collected, within the frame of disease exploration and with informed consent, from 25 patients who consulted at the Allergology Outpatient Clinic of the Centre Medical de l'Institut Pasteur between May and September 2007. These were 5 patients with atopic dermatitis, 7 patients with rhinitis, 6 patients with bronchial asthma and 7 patients with chronic urticaria (Table I). Atopic dermatitis fulfilled the clinical criteria of Hanifin (Hanifin et al 1980 Acta Dermatol. Venerol. (Suppl.) 92:44-47, Hanifin, J. M. 1984. Annals of allergy 52:386-395). Its severity was assessed by the Scoring Atopic Dermatitis (SCORAD) index (Kunz et al 1997. Dermatology (Basel, Switzerland) 195:10-19). Rhinitis and allergic asthma were classified according to the guidelines of the Allergic Rhinitis and its Impact on Asthma (ARIA) (Bachert, C., and P. van Cauwenberge. 2003. Chemical immunology and allergy 82:119-126) and of the Global Initiative for Asthma (GINA) (Bousquet et al 2007 Allergy 62:102-112). Chronic urticaria was defined as recurrent episodes of everyday hives for more than 6 months with no identified trigger. Three chronic urticaria patients had an associated autoimmune thyroiditis. All patients underwent skin prick tests with common aeroallergens. No patient received systemic immunosuppressive or corticosteroid treatment. The investigation was approved by the Biomedical Research Committee of the Institut Pasteur.

TABLE I
Patients included in the study

| | | | | | | Total IgE |
|---|---|---|---|---|---|---|
| Patient | Age | Sex | Disease | Allergens | Severity | (kU/l) |
| 1 | 21 | F | Atopic dermatitis | Polysensitized | Moderate | 402 |
| 2 | 26 | F | Atopic dermatitis | Polysensitized | Moderate | 4620 |
| 3 | 68 | M | Atopic dermatitis | Polysensitized | Moderate | 16556 |
| 4 | 30 | F | Atopic dermatitis | Polysensitized | Moderate | 2454 |
| 5 | 13 | F | Atopic dermatitis | Polysensitized | Severe | 4273 |
| 6 | 31 | M | Chronic urticaria | None | Moderate | 57 |
| 7 | 57 | M | Chronic urticaria* | None | Moderate | 105 |
| 8 | 26 | F | Chronic urticaria | None | Moderate | 126 |
| 9 | 26 | F | Chronic urticaria | None | Dermographia | 266 |
| 10 | 48 | M | Chronic urticaria | None | Moderate | 128 |
| 11 | 63 | F | Chronic urticaria* | None | Moderate | 158 |
| 12 | 65 | F | Chronic urticaria* | None | Moderate | 3 |
| 13 | 31 | M | Rhinitis | Pollen (grass, birch) | Moderate | 128 |
| 14 | 53 | M | Rhinitis | Pollen (grass, birch) | Moderate | 200 |
| 15 | 41 | F | Rhinitis | Mites, cat | Moderate | 146 |
| 16 | 36 | F | Rhinitis | Pollen (grass, birch), mites | Moderate | 140 |
| 17 | 25 | M | Rhinitis | Mites, cat | Moderate | 137 |
| 18 | 28 | M | Rhinitis | Pollen (grass) | Moderate | 609 |
| 19 | 21 | F | Rhinitis | Pollen (grass, birch) | Moderate | 183 |
| 20 | 19 | F | Asthma | Pollen (grass) | Persistent/moderate | 628 |
| 21 | 68 | F | Asthma | Mites | Persistent/moderate | 110 |
| 22 | 46 | F | Asthma | Pollen (grass, birch), cat | Persistent/moderate | 684 |
| 23 | 45 | M | Asthma | Mites | Persistent/moderate | 98 |
| 24 | 58 | F | Asthma | Pollen (grass), mites | Persistent/moderate | 117 |
| 25 | 47 | M | Asthma | mites | Persistent/moderate | 60 |

*associated with auto-immune thyroiditis

Serum IgE levels. Total serum IgE was measured by the enzyme immunoassay-based ACCESS kit (Beckman-CoulterVillepinte, France).

Antibodies. A) Fluorochrome-conjugated antibodies: Antibodies and corresponding isotype controls used for immunofluorescence analysis are listed in Table II. Phycoerythrin (PE)-conjugated CD3, CD14, CD19, CD24, CD56, MOPC21 and G155-178, and Fluorescein isothiocyanate (FITC)-conjugated CD16, CD32, MOPC21 and 27-35 were purchased from BD-Pharmingen (Le-Pont-de-Claix, France). PE-conjugated CD203c and 679.1Mc7 were from Beckman-Coulter (Marseille, France) Allophycocyanin (APC)-conjugated CD16 and control mouse monoclonal IgG1 were from Serotec (Düsseldorf, Germany). FITC-conjugated polyclonal Goat anti-human IgE was from Sigma-Aldrich (Saint-Quentin Fallavier, France) and FITC-conjugated normal polyclonal Goat IgG from Jackson ImmunoResearch (West Grove, Pa.). B) Nonconjugated antibodies: Polyclonal Rabbit anti-human IgE was from Dako Cytomation (Glostrup, Denmark). F(ab′)₂ fragments of Rabbit anti-mouse IgG (RAM) were from Jackson ImmunoResearch. 3G8 F(ab′)₂ fragments were prepared by pepsin digestion of 3G8 IgG antibodies purified from hybridoma supernatant by affinity chromatography on Protein G sepharose (Amersham Biosciences, Saclay, France).

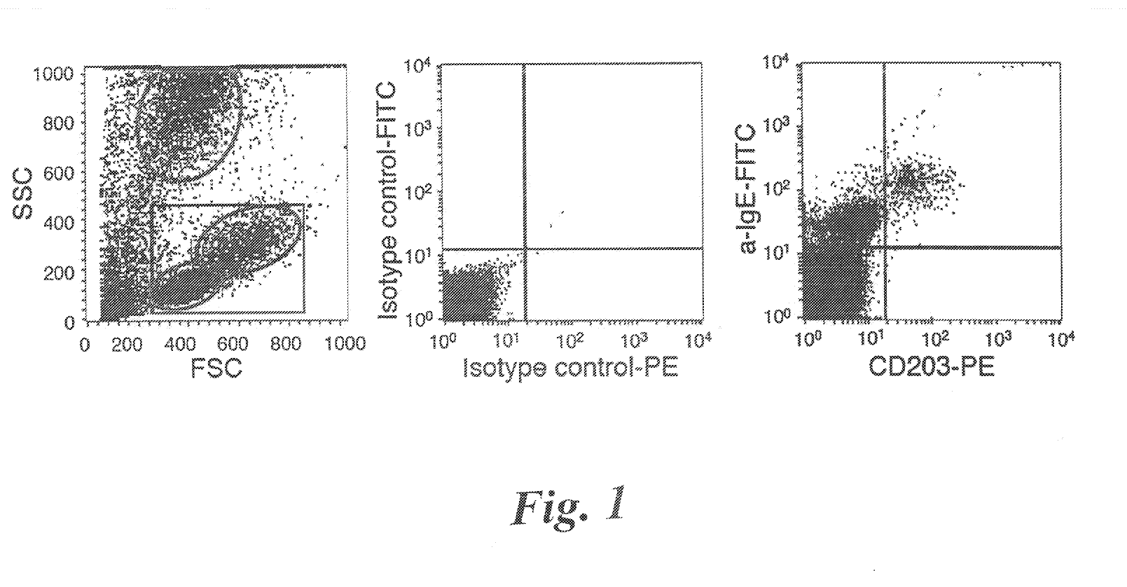

Immunofluorescence. Whole blood cells were depleted of Red Blood Cells (RBCs) by hypotonic lysis in buffer containing 77 mM NH₄Cl, 3.6 mM K₂CO₃ and 0.4 mM EDTA, and washed in phosphate-buffered saline containing 0.2% bovine serum albumin (PBS-BSA). Cells were incubated with PE-conjugated, FITC-conjugated and/or APC-conjugated antibodies for 15 min at 0° C. Cell fluorescence was analyzed by flow cytometry in gates shown in FIG. 1, using a FACScalibur (BD Bioscience, Le Pont de Claix, France). A constant number of 1,000 CD203⁺ basophils was acquired.

TABLE II
Antibodies used for immunofluorescence analysis

| Fluorochrome | Isotype | Ab (clone) | Isotype control |
|---|---|---|---|
| PE | Mouse IgG1 | CD56 (B159) | MOPC-21 |
| | Mouse IgG1 | CD19 (HIB19) | MOPC-21 |
| | Mouse IgG1 | CD203C (97A6) | 679.1Mc7 |
| | Mouse IgG2a | CD14 (M5E2) | G155-178 |
| | Mouse IgG2a | CD3 (HIT3a) | G155-178 |
| | Mouse IgG2a | CD24 (ML5) | G155-178 |
| FITC | Mouse IgG1 | CD16 (3G8) | MOPC-21 |
| | Mouse IgG2b | CD32 (FLI8.26) | 27-35 |
| | Goat IgG | a-human IgE | Normal Goat IgG |
| APC | Mouse IgG1 | CD16 (3G8) | Mouse monoclonal |
| | | | IgG1 |

PI-PLC digestion. RBC-depleted blood cells were incubated with 0.5 IU/ml Phosphatidylinositol-specific phospholipase C (PI-PLC) (Sigma-Aldrich, Saint-Louis, Mo.) for 30 min at 37° C. The enzymatic reaction was stopped by washing cells in cold PBS-BSA. Cells were stained with fluorescent antibodies and analyzed by flow cytometry.

Basophil activation. A) anti-IgE-induced: Blood cells were incubated with 10 μg/ml anti-IgE antibodies for 30 min at 37° C. before RBCs were depleted by hypotonic lysis. They were then stained with PE-CD203c, and fluorescence was analyzed by flow cytometry. B) CD16-induced: RBC-depleted cells were incubated with 10 μg/ml 3G8 F(ab′)₂ fragments for 30 min at 37° C., washed, and incubated with 30 μg/ml RAM F(ab′)₂ for 30 min at 37° C. Stimulation was stopped by washing cells in cold PBS-BSA. Cells were stained with PE-CD203c, and analyzed by flow cytometry.

Statistical analysis. Data were analyzed with the Statistical Analysis System package for Macintosh (SAS Institute, Campus Drive Cary, N.C.). A nonparametric Mann-Whitney U test was used to compare normal donors and patients.

Results

Human Basophils Co-Express FcεRI, FcγRII (CD32) and FcγRIII (CD16)

When analyzed by flow cytometry, RBC-depleted blood cells separate into three main populations with distinct forward and side scatter values (FSC and SSC, respectively) (FIG. 1). A population with a high SSC and an intermediate FSC contains polymorphonuclear neutrophils. A population with a low SSC and an intermediate FSC contains lymphocytes. A population with an low-to-intermediate SSC and a high FSC contains monocytes. Basophils are dispersed among lymphocytes and monocytes. They can be identified as cells doubly labeled with anti-IgE and CD203 in a gate encompassing these two populations (FIG. 1). IgE⁺ CD203⁺ cells represent 0.5-1% of total white blood cells (0.97% in the experiment shown in FIG. 1). Noticeably, IgE cells included CD203⁺ and CD203⁻ cells, whereas all CD203⁺ cells were IgE⁺. CD203 alone is therefore sufficient to identify blood basophils.

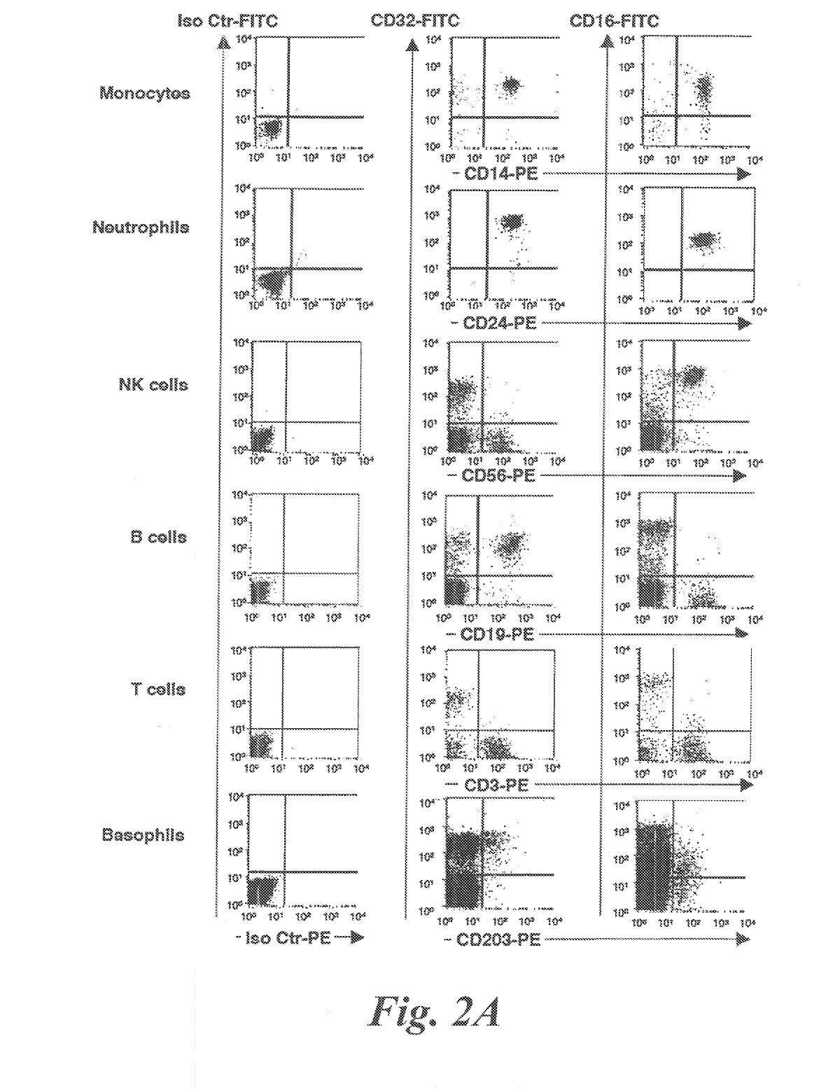

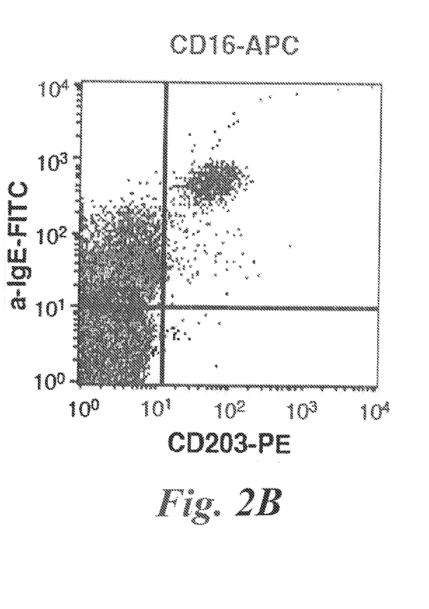

We first examined the expression of the two classes of low-affinity IgG receptors, FcγRII (CD32) and FcγRIII (CD16), on basophils and, as positive and negative controls, on other white blood cells. RBC-depleted blood cells from normal donors were doubly stained with PE-labeled cell-type specific antibodies and with CD32-FITC or CD16-FITC, and fluorescence was analyzed in cells gated as shown in FIG. 1. As expected, monocytes, identified by CD14, and neutrophils, identified by CD24, expressed both CD32 and CD16. NK cells, identified by CD56, expressed CD16, but not CD32, whereas B cells, identified by CD19, expressed CD32, but not CD16. T cells, identified by CD3, expressed neither CD32 nor CD16, except a minor CD32⁻ CD16⁺ subpopulation, probably corresponding to NKT cells. As expected, basophils, identified by CD203, expressed CD32. Unexpectedly, a significant proportion of basophils also expressed detectable levels of CD16 (FIG. 2A). Triple labeling with CD203-PE, CD16-APC and anti-IgE-FITC confirmed that CD203⁺ IgE⁺ cells were indeed stained by CD16 (FIG. 2B). A proportion of basophils from normal donors therefore express not only FcεRI and FcγRII (CD32), but also FcγRIII (CD16).

FcγRIII Expressed by Human Basophils is CD16B

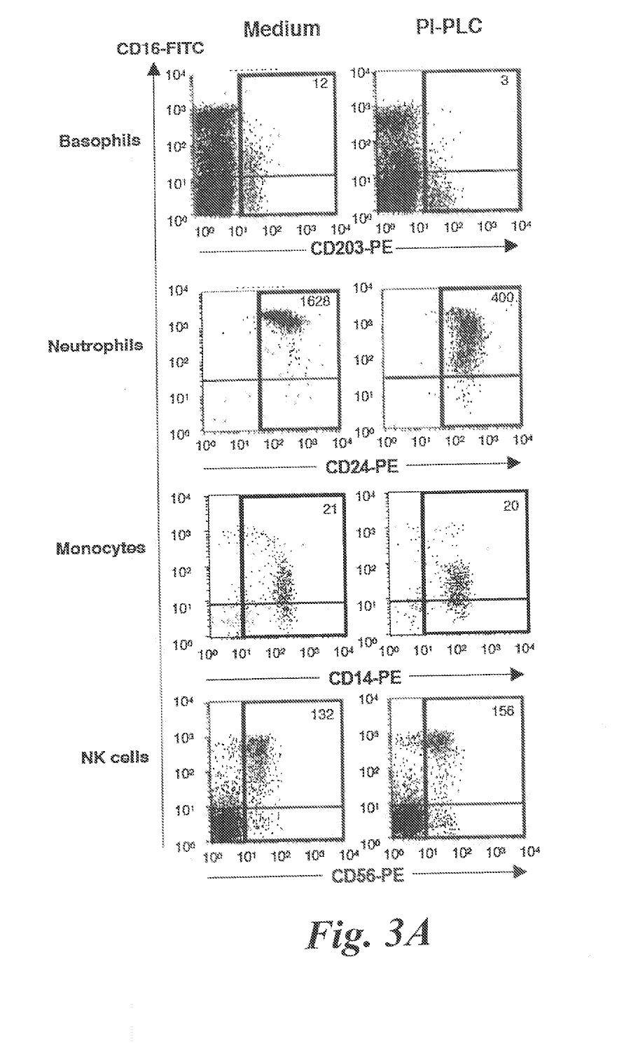

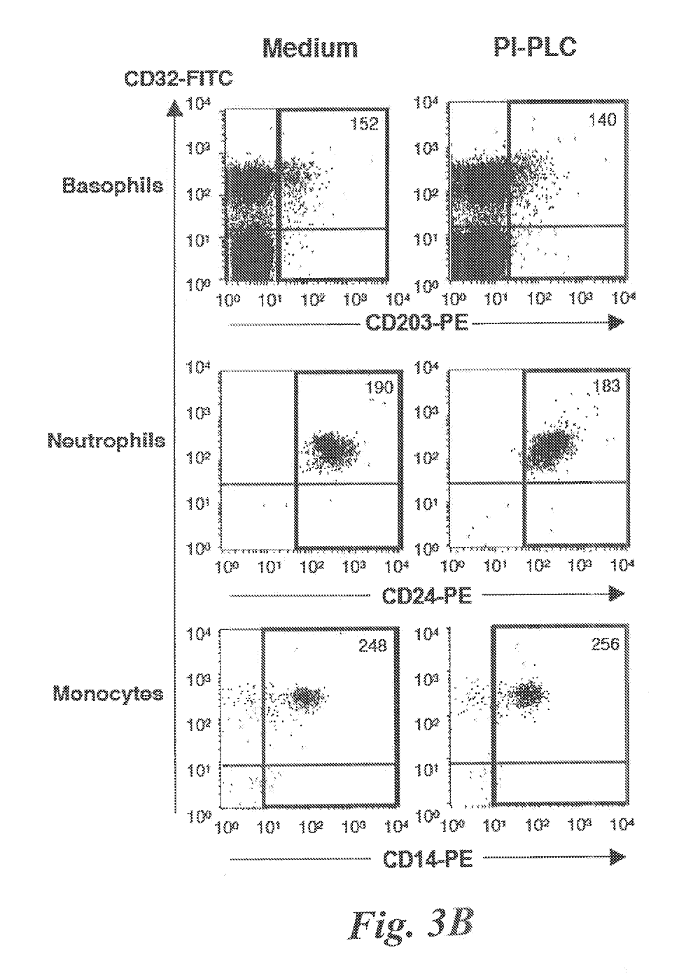

FcγRIIIA and FcγRIIIB can be distinguished by their differential sensitivity to phopshatidylinositol-specific phospholipase C (PI-PLC). GPI-anchored FcγRIIIB, but not transmembrane FcγRIIIA, are indeed cleaved by PI-PLC (29, 30). To determine which isoform(s) of FcγRIII is (are) expressed by basophils, RBC-depleted blood cells were therefore treated or not treated with PI-PLC, and doubly stained with PE-labeled cell-type specific antibodies and with CD16-FITC or CD32-FITC. PI-PLC treatment decreased the CD16 staining not only of CD24⁺ neutrophils, as expected, but also of CD203⁺ basophils (FIG. 3A). Noticeably the mean fluorescence intensity (MFI) of CD16 staining was comparably reduced (4-fold) in both cell types, and it was virtually abrogated in basophils. By contrast, PI-PLC treatment affected neither the CD16 staining of CD14⁺ monocytes and CD56⁺ NK cells (FIG. 3A), which express FcγRIIIA but not FcγRIIIB, nor the CD32 staining of CD203⁺ basophils, CD24⁺ neutrophils and CD14⁺ monocytes (FIG. 3B). Basophils from normal donors therefore express FcγRIIIB (CD16B) but not FcγRIIIA (CD16A).

CD16B Expression is Down Regulated Upon Basophil Activation

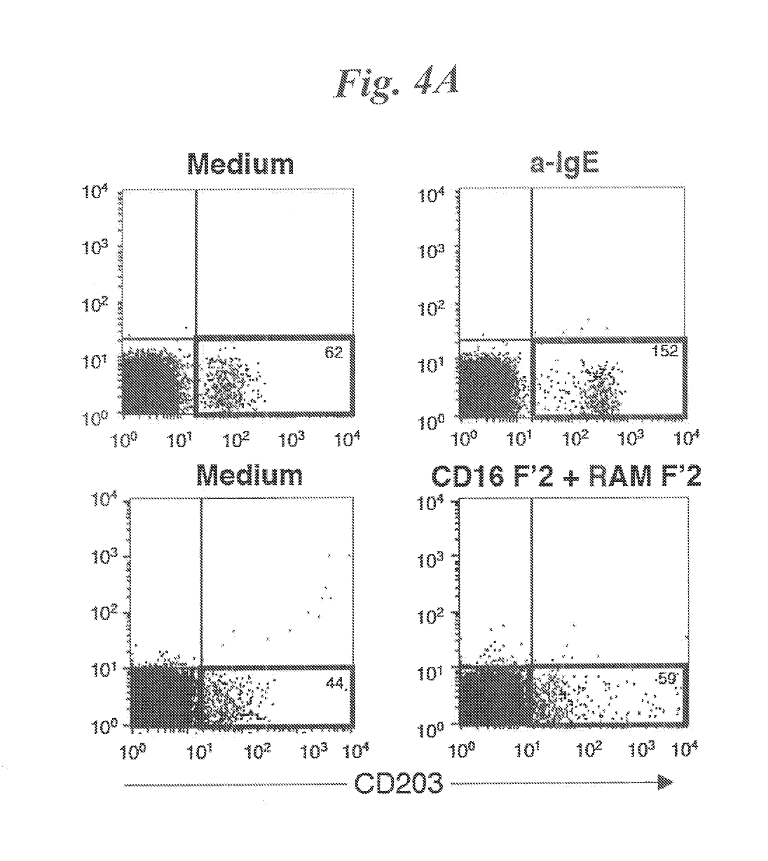

To determine whether the engagement of FcγRIIIB could activate basophils, CD203 expression was monitored on RBC-depleted blood cells stimulated with CD16 F(ab′)₂ and rabbit anti-mouse Ig (RAM) F(ab′)₂. Whereas FcεRI aggregation by anti-IgE markedly upregulated CD203 expression, no or a minor CD203 upregulation was seen following FcγRIIIB aggregation (FIG. 4A). The engagement of CD16 therefore does not or poorly activates basophils, when assessed by CD203 upregulation.

Cell activation was reported to decrease the expression of CD16B by neutrophils (31). CD16 and CD203 expression were therefore examined on basophils from 8 normal donors, before and after stimulation with anti-IgE. The MFI of CD203 staining increased similarly on basophils from all 8 donors, following anti-IgE stimulation (Median MFI increase: 2.8-fold). The MFI of CD16 staining differed on nonstimulated basophils from the same donors. Following anti-IgE stimulation, CD16 expression markedly dropped on basophils from 4 of the 5 donors with a high expression, and decreased on basophils from the 3 donors with a weak expression (FIG. 4B) (Median MFI decrease: 2-fold). Anti-IgE-induced basophil activation therefore correlates with a decreased CD16 expression.

CD16B⁺ Basophils are Found, in Variable Numbers, in all Donors

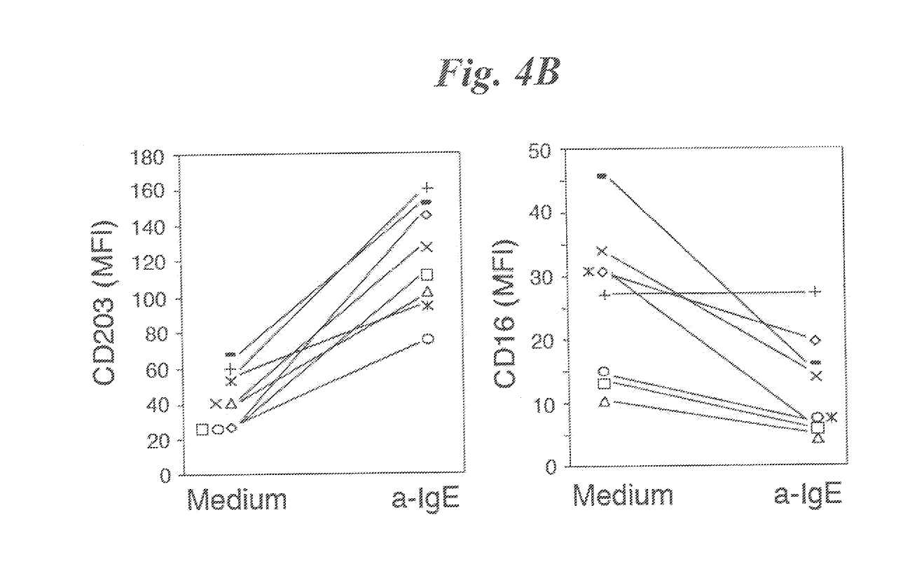

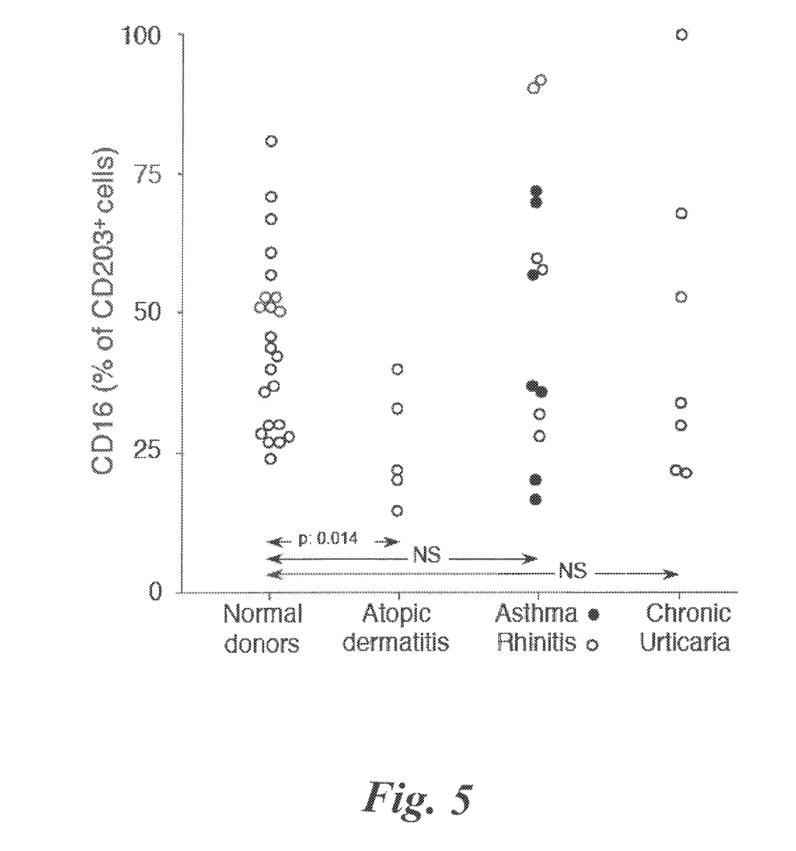

To determine whether CD16 expression by normal basophils is a rule or an exception, RBC-depleted blood from 23 normal donors were doubly stained with CD203 and CD16. As expected, the percentage of CD203⁺ blood cells stained by CD16 over the background varied from donor to donor. Between 24 and 81% basophils (median 44%) were detectably stained with CD16. Noticeably, all 23 normal donors tested had CD16⁺ basophils (FIG. 5). CD16 is therefore expressed by at least one fourth of blood basophils in normal individuals.

CD16B Expression is Decreased on Basophils from Atopic Dermatitis Patients

We next compared CD16 expression on basophils from normal donors and from 25 patients with diseases commonly seen in allergy practice. These were 5 patients with an allergic skin disease (atopic dermatitis), 13 patients with allergic respiratory diseases (rhinitis and asthma), and 7 patients with a nonallergic skin disease (chronic urticaria) (Table I). Like normal subjects, all 25 patients had CD16⁺ basophils. The proportion of CD16⁺ basophils, however, varied more widely in patients than in normal donors (from 12 to 100%). It did not differ statistically from normal donors in respiratory allergy patients (median 57%) and in chronic urticaria patients (median 34%). It was significantly lower (p=0.0142) in atopic dermatitis patients (median 22%) (FIG. 5).

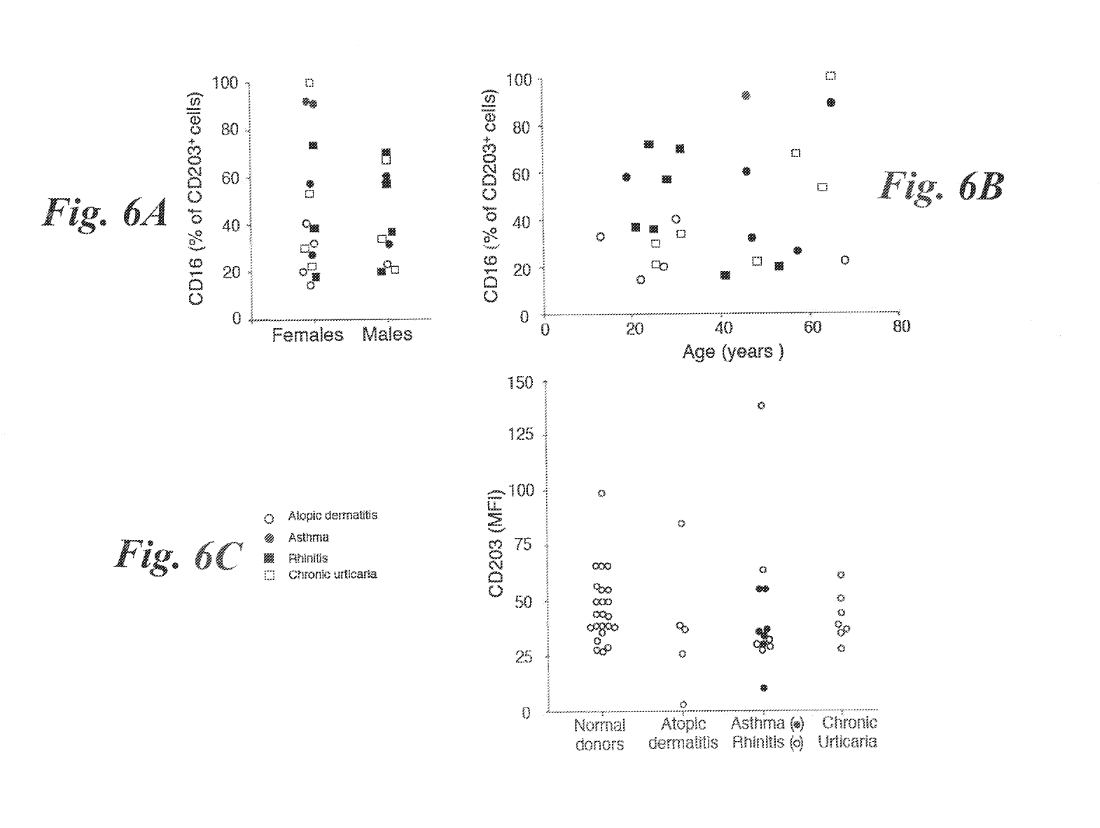

Four of the 5 atopic dermatitis patients were females. The percentage of CD16⁺ basophils was however not lower in females than in males patients (FIG. 6A). In average, dermatitis patients were younger than other patients. The percentage of CD16⁺ basophils was however not correlated with the age of patients (FIG. 6B). Because we observed that CD16 expression was decreased upon basophil activation, we investigated whether basophils might be constitutively activated in atopic dermatitis patients, by examining the intensity of CD203 staining in the three groups of patients. The MFI of CD203 staining was not different in atopic dermatitis patients, compared with normal donors (FIG. 6C).

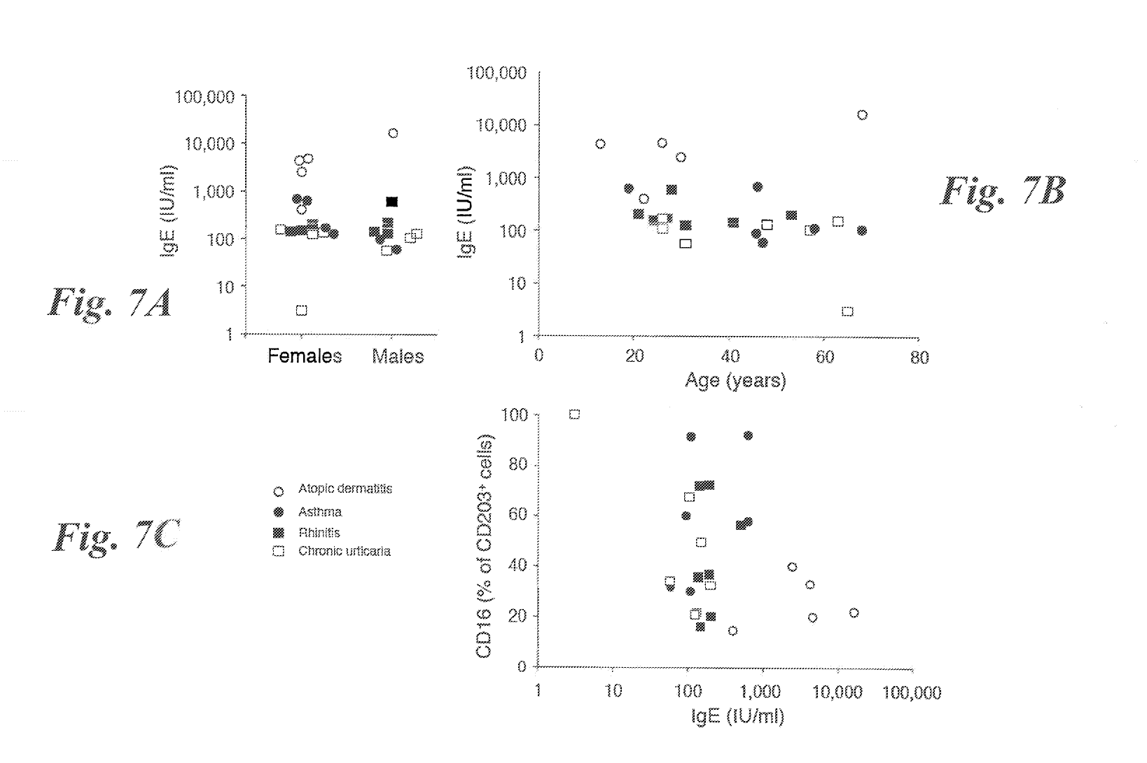

A hallmark of atopic dermatitis is a high serum IgE level and, indeed, the serum concentration of IgE was markedly higher in atopic dermatitis patients (median 4273 IU/ml) than in allergic respiratory disease patients (median 147 IU/ml) and in chronic urticaria patients (median 126 IU/ml) (Table I). IgE concentration was correlated neither with the gender (FIG. 7A) nor with the age of patients (FIG. 7B). The percentage of CD16⁺ basophils, was not correlated either with serum IgE levels (FIG. 7C).

Discussion

Results reported here challenge two widely accepted notions. First, human basophils are considered as CD16-negative cells (Reimer et al 2006 Allergy 61:1063-1070, Takahashi, et al 1993 J Immunol Methods 162:17-21, Han et al 1999 Cytometry 37:178-183). We show that a significant proportion of blood basophils express CD16 in all donors, whether normal individuals or patients. Second, the GPI-anchored FcγRIIIB (CD16B) is considered as being solely expressed by human neutrophils (Li et al 1996 J. Exp. Med. 183:1259-1263). We show that CD16B is also expressed by basophils. Interestingly, we also show that the expression of CD16B decreases on basophils upon anti-IgE-induced activation and that the percentage of CD16B-expressing basophils is lower in atopic dermatitis patients than in normal donors. These findings may have practical, fundamental and clinical implications.

CD16 is typically expressed by blood neutrophils, monocytes, NK and NKT cells, but not by B cells, T cells and basophils. We found that at least 24%, and up to 81% basophils from normal donors were detectably labeled by CD16. Likewise, between 12 and 100% basophils from allergic (and nonallergic) patients detectably expressed CD16. Several reasons may explain why CD16 was not previously observed on basophils. One is that reference works in which basophils were first phenotyped were done at a time when human FcγR isoforms had not yet been all identified and with mAbs whose specificity for FcγR subtypes was unclear (de Boer, M., and D. Roos. 1986. J Immunol 136:3447-3454; Stain, et al 1987 Blood 70:1872-1879; Toba et al 1999 Cytometry 35:249-259). Another reason is the much lower expression of CD16 by basophils than by other CD16⁺ blood cells. MFI values of CD16 were indeed 10-100-fold lower for basophils than for monocytes, NK cells or neutrophils. Techniques with a relatively low sensitivity, which would be sufficient to brightly label these cells, may not or hardly label basophils. One may have interpreted a weak basophil labeling as background labeling and have neglected it. Finally, CD16 may not have been seen in sorted basophil preparations because these basophils did not express CD16. Basophil enrichment techniques use magnetic beads coated with a cocktail of mAbs against membrane molecules that are not expressed by basophils. As basophils were described to express CD13, CD22, CD32, CD33, CD123 and CD203c, but not CD2, CD3, CD14, CD15, CD16, CD19, CD21, CD64 and HLA-DR (Reimer et al 2006. Allergy 61:1063-1070; Takahashi et al 1993 J Immunol Methods 162:17-21; Han et al 1999 Cytometry 37:178-183), negative selection procedures used to enrich basophils from white blood cells include CD16 (Takahashi et al 1993 J Immunol Methods 162:17-21, Gibbs et al 1997 Inflamm Res 46:137-142) among other mAbs. Even though CD16 expression is low on basophils, one cannot exclude that enrichment procedures in which CD16⁺ cells are discarded may remove a significant proportion of basophils. This would introduce a bias in sorted basophils. It may also decrease the yield of these techniques.

On the basis of the differential sensitivity of CD16A and CD16B to PI-PLC, we found that basophils express the GPI-anchored FcγRIIIB (CD16B), but not the transmembrane, FcRγ-associated FcγRIIIA (CD16A). PI-PLC treatment of blood cells indeed reduced similarly the staining of basophils and neutrophils, but not of monocytes or NK cells, by CD16, and had no detectable effect on the staining of any blood cells, including basophils, by CD32. The CD16 staining of basophils was reduced below the background level in PI-PLC-treated cells, indicating that basophils do not express CD16A. It follows that the expression of CD16B is not restricted to neutrophils as previously thought.

The biological functions of CD16B are poorly understood. When expressed in transfectants and aggregated by appropriate plurivalent ligands, FcγRIIIB did not trigger activation signals (Nagarajan et al 1995 J Biol Chem 270:25762-25770). FcγRIIIB aggregation was, however, reported to trigger the production of H₂O₂ and an increased intracellular Ca²⁺ concentration in neutrophils (Hundt, M., and R. E. Schmidt. 1992. Eur J Immunol 22:811-816). Neutrophil CD16B were also found to trigger degranulation (Unkeless et al 1995 Semin Immunol 7:37-44), and to activate Tec (Fernandes et al 2005 J Leukoc Biol 78:524-532) and Syk (Fernandes et al 2006 Biochem J 393:351-359) protein tyrosine kinases. We failed to induce a significant CD203 upregulation when aggregating CD16B on basophils under similar conditions. CD203 upregulation is associated with exocytosis (Buhring et al 2004 Int Arch Allergy Immunol 133:317-329). Whether the engagement of CD16B can induce responses of basophils other than degranulation, e.g. cytokine synthesis/secretion, remains to be investigated. In any case, our finding that basophils do not degranulate in response to CD16 engagement supports the conclusion that basophils do not express CD16A.

Like other GPI-anchored molecules, CD16B constitutively resides in cholesterol- and sphyngolipid-rich plasma membrane microdomains or lipid rafts (Fernandes et al 2006 Biochem J 393:351-359; Brown, D. A., and E. London. 2000 J. Biol. Chem. 275:17221-17224). Signaling molecules, including Src-family protein tyrosine kinases (Young et al 2003 J Biol Chem 278:20746-20752), and the transmembrane adapter LAT (Zhang et al 1998 Immunity 9:239-246) are also located in lipid rafts, and the aggregation of lipid raft-associated gangliosides was reported to trigger activation signals (Valensin et al 2002 Eur J Immunol 32:435-446). Besides, CD16B was proposed to synergize with activating FcRs (Unkeless, et al 1995 Semin Immunol 7:37-44) by facilitating the binding of IgG immune complexes to other FcγRs on the same cell (Fernandes et al 2006 Biochem J 393:351-359, Moser et al 1995 The Journal of laboratory and clinical medicine 126:588-596). CD16B could also compete with other FcγRs for binding immune complexes. Whether CD16B may enhance or decrease cell signaling by other basophil FcγRs needs to be determined. The expression of several low-affinity IgG receptors with different signaling properties may indeed explain why no clearcut conclusion was drawn, as for the ability of IgG immune complexes to activate basophils (Van der Zee, J. S., and R. C. Aalberse. 1991 Eur Respir J Suppl 13:91s-96s).

As seen previously on activated neutrophils (Huizing a et al 1988 Nature (Lond.) 333:667-669), the expression of CD16B was downregulated on activated basophils. CD16B downregulation was proposed to result from the cleavage of the receptor by proteases released and/or activated during neutrophil activation (Sautès, C. 1997. Soluble Fc receptors. Landes, Austin, Tex.). Basophils may similarly release and/or activate proteases when activated. Whereas mast cell proteases are well characterized and used as mast cell type-specific markers (Pejler et al 2007 Adv Immunol 95:167-255), basophil proteases are poorly known. Murine basophils were however described to express mMCP-8. This protease is related to T cell granzymes and to the neutrophil cathepsin G, rather than to mast cell tryptases or chimases (Lunderius, C., and L. Hellman. 2001 Immunogenetics 53:225-232).

Interestingly, the percentage of CD16⁺ basophils was significantly lower in atopic dermatitis patients than in normal donors, but not in chronic urticaria patients, in allergic rhinitis patients or in allergic asthma patients. A decreased expression of CD16B by basophils was therefore observed in an allergic skin disease, but neither in a nonallergic skin disease nor in two allergic respiratory diseases. Noticeably, CD16B expression was not decreased on neutrophils from atopic dermatitis patients (not shown), suggesting that CD16B downregulation selectively affects basophils. CD16B downregulation was not correlated with CD203 upregulation on basophils from atopic dermatitis patients, suggesting that it was not associated with a possible chronic basophil activation. The percentages of CD16⁺ basophils was not correlated with serum IgE levels which were markedly elevated in atopic dermatitis patients. IgE-bearing antigen-presenting cells, T cells, mast cells, eosinophils and keratinocytes concur to generate atopic dermatitis lesions (Boguniewicz, M., and D. Y. Leung. 2006 J Allergy Clin Immunol 117:S475-480). Interestingly, basophils were observed in skin biopsies after allergen challenge (Irani et al 1998 J Allergy Clin Immunol 101:354-362). The biological significance of the decreased frequency of CD16⁺ basophils in atopic dermatitis patients is not known. Whether it is a cause or a consequence in this disease and whether it has pathological consequences need to be investigated. It nevertheless supports the idea that, although a skin disease, atopic dermatitis is a systemic disease rather than a disease of the skin.

Obviously, numerous modifications and variations of the present invention are possible in light of the above teachings. It is therefore to be understood that within the scope of the appended claims, the invention may be practiced otherwise than as specifically described herein.

1. A method for enriching basophils in a blood sample, comprising
(a) contacting the sample with an antibody cocktail which comprises antibodies that specifically bind to non-basophil cells in the sample and does not include any anti-CD16 antibodies,
(b) separating the non-basophil cells from the sample to obtain a blood sample enriched with basophils.

2. The method of claim 1, wherein the antibody cocktail comprises at least one antibody which specifically binds to neutrophils, at least one antibody which specifically binds to natural killer cells, at least one antibody which specifically binds to monocytes, at least one antibody which specifically binds to dendritic cells, at least one antibody which specifically binds to T lymphocytes, at least one antibody which specifically binds to B lymphocytes, and at least one antibody which specifically binds to eosinophils.

3. The method of claim 1, wherein the antibody cocktail further comprises at least one antibody which specifically binds to erythrocytes and at least one antibody which specifically binds to platelets.

4. The method of claim 1, wherein the antibody cocktail comprises
at least one antibody specific for CD14,
at least one antibody specific for CD24, and
at least one antibody specific for CD56.

5. The method of claim 1, wherein the antibody cocktail comprises:
at least one antibody specific for CD3,
at least one antibody specific for CD4,
at least one antibody specific for CD7,
at least one antibody specific for CD14,
at least one antibody specific for CD15,
at least one antibody specific for CD24,
at least one antibody specific for CD36,
at least one antibody specific for CD45RA,
at least one antibody specific for HLA-DR,
at least one antibody specific for CD56, and
at least one antibody specific for CD235a.

6. The method of any one of claim 1, wherein the antibody cocktail comprises:
at least one antibody specific for CD2,
at least one antibody specific for CD3,
at least one antibody specific for CD14,
at least one antibody specific for CD15,
at least one antibody specific for CD19,
at least one antibody specific for CD24,
at least one antibody specific for CD34,
at least one antibody specific for CD36,
at least one antibody specific for CD45RA,
at least one antibody specific for CD56, and
at least one antibody specific for CD235a.

7. The method of claim 1, wherein the antibodies are monoclonal antibodies.

8. The method of claim 1, wherein the antibodies are conjugated to magnetic beads.

9. The method of claim 8, wherein the separating comprises applying a magnetic field to deplete the blood sample of non-basophil cells.

10. The method of claim 1, wherein the antibodies are conjugated to biotin, and the separating comprises contacting the blood sample with anti-biotin antibodies conjugated to a magnetic bead and applying a magnetic field to deplete the sample of non-basophil cells.

11. The method of claim 1, wherein the blood sample is peripheral blood, buffy coat, peripheral blood mononuclear cells (PBMC), or umbilical cord blood.

12. The method of claim 1, further comprising before (a), the blood sample is pre-enriched in mononuclear cells by density gradient centrifugation, counter current centrifugal elutriation, erythrocytes lysis, Percoll gradients, or a combination thereof.

13. The method of claim 1, further comprising after (b), evaluating the purity of the enriched basophils.

14. The method of claim 1, wherein the anti-CD16 antibodies are specific for CD16B.

15. A kit for enriching basophils in a blood sample, comprising antibodies that specifically bind to non-basophil cells but excluding any anti-CD16 antibody.

16. The kit of claim 15, wherein the anti-CD16 antibody is specific for a CD16B isoform.

17. The kit of claim 15, comprising at least one antibody specific for CD14, at least one antibody specific for CD24, and at least one antibody specific for CD56.

18. The kit of claim 15, comprising at least one antibody specific for CD3, at least one antibody specific for CD4, at least one antibody specific for CD7, at least one antibody specific for CD14, at least one antibody specific for CD15, at least one antibody specific for CD24, at least one antibody specific for CD36, at least one antibody specific for CD45RA, at least one antibody specific for HLA-DR, at least one antibody specific for CD56, and at least one antibody specific for CD235a.

19. The kit of claim 15, comprising at least one antibody specific for CD2, at least one antibody specific for CD3, at least one antibody specific for CD14, at least one antibody specific for CD15, at least one antibody specific for CD19, at least one antibody specific for CD24, at least one antibody specific for CD34, at least one antibody specific for CD36, at least one antibody specific for CD45RA, at least one antibody specific for CD56, and at least one antibody specific for CD235a.

20. The kit of claim 15, wherein the antibodies are monoclonal antibodies.

21. The kit of claim 15, wherein the antibodies are conjugated to magnetic beads.

22. The kit of claim 15, wherein the antibodies are conjugated to biotin.

23. The kit of claim 15, wherein the antibodies are physically separated or cocktailed to comprise two or more antibodies.
